(19) United States
(12) Patent Application Publication
(10) Pub. No.: US 2026/0083681 A1
(43) Pub. Date: Mar. 26, 2026

(54) FORMULATIONS OF SPRAY DRIED LIPID NANOPARTICLES

(71) Applicant: Serán BioScience, LLC, Bend, OR (US)

(72) Inventors: Mark Kastantin, Bend, OR (US); Dan Smithey, Bend, OR (US); Erica Schlesinger, Sisters, OR (US); Ryan Mowry, Bend, OR (US)

(21) Appl. No.: 19/330,723

(22) Filed: Sep. 16, 2025

Related U.S. Application Data
(60) Provisional application No. 63/698,168, filed on Sep. 24, 2024.

(51) Int. Cl. A61K 9/51; A61K 9/00; A61K 31/7105

(52) U.S. Cl. CPC A61K 9/5123; A61K 9/0019; A61K 9/5161; A61K 9/5192; A61K 31/7105

(57) ABSTRACT

A formulation is provided for a spray dried lipid nanoparticle matrix particles, including lipid nanoparticles, a buffer system, and a barrier matrix stabilizer, wherein the barrier matrix stabilizer is included in a solid weight percent based on a maximum osmolarity and a maximum acceptable injectable volume of an injectable formulation comprising the spray dried lipid nanoparticle matrix particles.

### CROSS-REFERENCE TO RELATED APPLICATIONS

The present application claims priority to U.S. Provisional Application No. 63/698,168 entitled “FORMULATIONS OF SPRAY DRIED LIPID NANOPARTICLES”, and filed on Sep. 24, 2024. The entire contents of the above mentioned application are hereby incorporated by reference for all purposes.

### FIELD

The present description relates generally to formulations for spray drying lipid nanoparticles.

### BACKGROUND/SUMMARY

A lipid nanoparticle (LNP) may be used to encapsulate a biologic active pharmaceutical ingredient (API) such as RNA, DNA, proteins, and the like. LNPs including the API payload may be prepared in a liquid and the liquid may be subsequently removed by spray drying. Removing the liquid may result in dried LNPs which may have extended long term stability for storage at ambient temperature compared to LNPs remaining in liquid suspension. Further, removing the liquid after preparation enables formulation of injectable suspensions of LNPs at clinically relevant dosages. However, to prepare the injectable suspension demands that the LNPs maintain desired physical attributes when dried and resuspended. Such physical attributes are linked to ability of the LNP to function as a drug delivery vehicle and include average particle size, particle size distribution, and encapsulation efficiency. Conventionally, spray drying excipients including small sugar or sugar alcohol molecules such as sucrose, trehalose, or mannitol which are approved for parenteral or pulmonary delivery are included in a spray drying formulation to increase physical stability of the LNPs. However, these excipients may not result in the desired physical attributes of the LNPs when resuspended. Stability of LNPs may, conventionally, be further increased by keeping the LNP concentration low relative to the stabilizing excipients, but this strategy may limit a dose in an injection of the LNPs given a maximum osmolarity of an injectable suspension.

The inventors have recognized the above issued and in one example, the issues described above may be at least partially addressed by a spray dried lipid nanoparticle matrix particles, comprising lipid nanoparticles; a buffer system; and a barrier matrix stabilizer, wherein the barrier matrix stabilizer is included in a solid weight percent based on a maximum osmolarity and a maximum acceptable injection volume of an injectable formulation comprising the spray dried lipid nanoparticle matrix particles. The spray dried lipid nanoparticle matrix particles may be stable as dry powders and maintain desired physical properties after resuspension. The barrier matrix stabilizer may be a high molecular weight, high glass transition temperature, hydrogen bonding molecule which may be included in an effective amount for stabilizer the spray dried lipid nanoparticle matrix particles.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows a flowchart of a method for preparing spray dried lipid nanoparticle (LNP) matrix particles.

FIG. 2A shows a flowchart of a method for determining solid composition of LNP spray drying matrix solution.

FIG. 2B shows a graph of solids weight percent of components of the LNP spray drying matrix solution as a function of maximum osmolarity.

FIG. 3 shows a chart of Z-average and polydispersity index (PDI) of lipid nanoparticle matrix solutions before spray drying and after drying/resuspension.

FIG. 4 shows a chart of Z-average and PDI pre spray drying, resuspended directly after spray drying, and resuspended 13 weeks after spray drying.

FIG. 5 shows a chart comparing encapsulation efficiency of lipid nanoparticles before and after spray drying.

FIG. 6 shows charts comparing Z-average and PDI and encapsulation efficiencies of lipid nanoparticle matrix solutions with HP-b-CD and lipid nanoparticle matrix solutions with trehalose before and after spray drying.

FIG. 7 shows additional charts comparing Z-average, PDI and encapsulation efficiencies for lipid nanoparticle matrix solutions including different amounts of lipid nanoparticles before and after spray drying.

### DETAILED DESCRIPTION

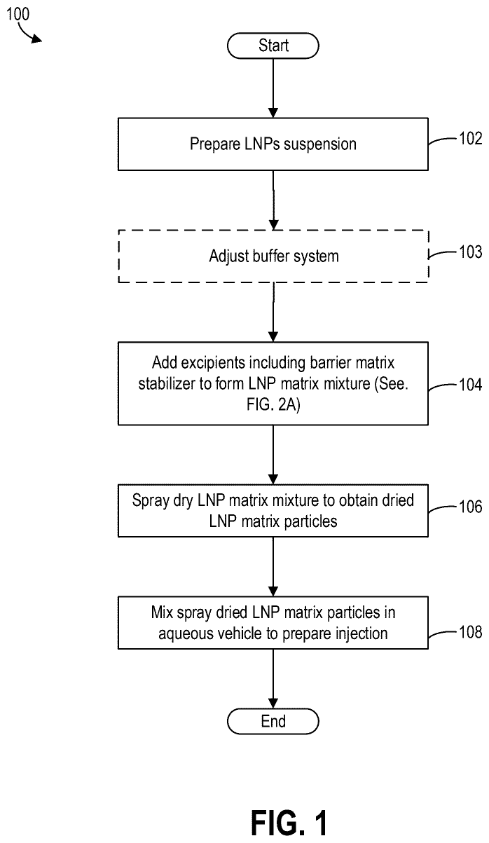

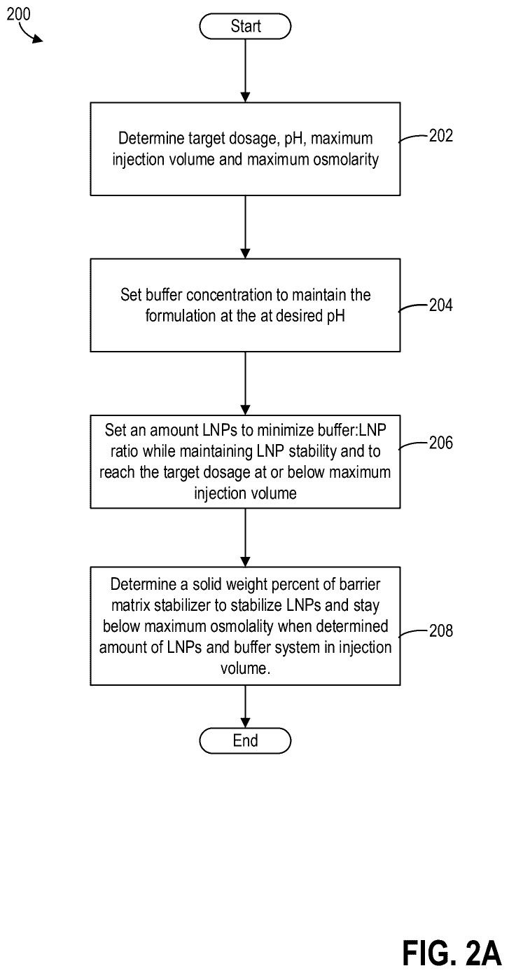

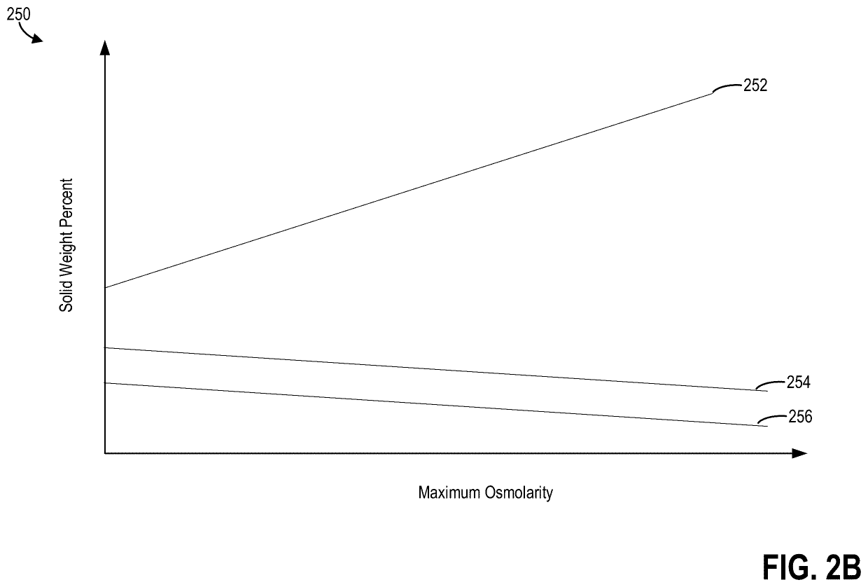

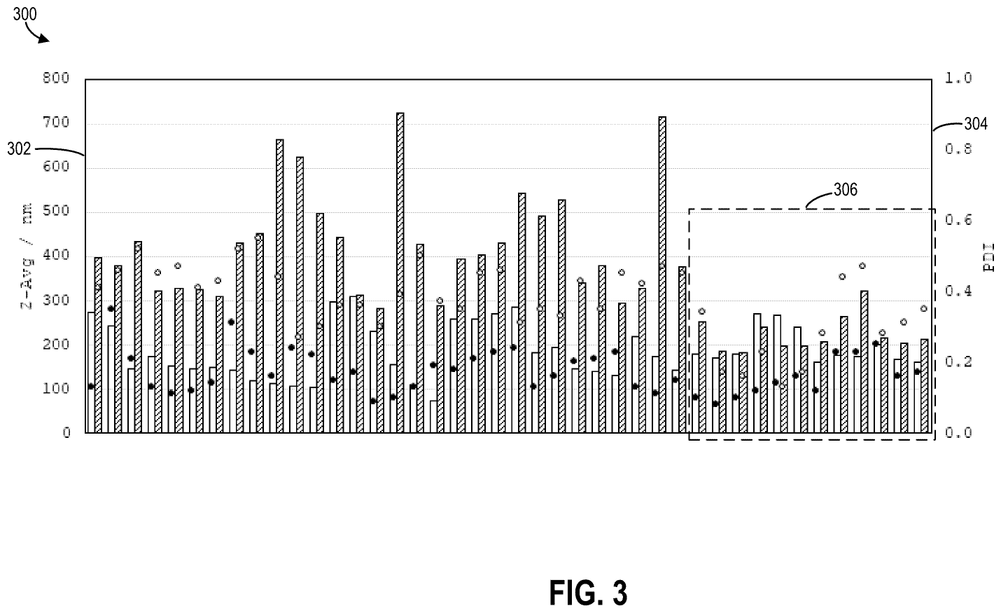

The following description relates to a formulation of a lipid nanoparticle (LNP) spray drying matrix solution and resulting spray dried LNP matrix particles. After forming the LNPs, a LNP spray drying matrix solution is formed and then spray dried to arrive at spray dried LNP matrix particles which may then be resuspended. FIG. 1 shows a flowchart of a method for preparing the LNP spray drying matrix solution and dried LNP matrix particles. A composition of the LNP spray drying matrix solution may affect physical properties of the LNP matrix particles when resuspended. Physical stability of spray dried LNP matrix particles may be increased by including a maximum amount of barrier matrix stabilizer. FIG. 2A shows a flowchart of a method for determining the maximum amount of barrier matrix stabilizer based on a target dosage, pH, injection volume, and osmolarity and FIG. 2B shows a graph of solids weight percent as function of target osmolarity. FIGS. 3-7 compare the physical properties of different LNP spray drying matrix solutions before and after spray drying.

Turning now to FIG. 1, a method for preparing dried LNP matrix particles and injectable formulations comprising the dried LNP matrix particles is shown. At 102, method 100 includes preparing a LNP suspension. The LNP suspension may be formed of a liquid and lipid nanoparticles encapsulating an active pharmaceutical ingredient (API). As one example, the liquid may be an aqueous/organic mixture. An organic solvent of the aqueous/organic mixture may include water and alcohols such as, but not limited to, methanol and ethanol. The aqueous component may include water at >25% or >50% by volume. The water included may be buffered water instead of pure water. In further examples, the organic solvent may additionally or alternatively include acetone, tetrahydrofuran (THF), among others. The lipid nanoparticles may be formed of a combination of lipids, cholesterol, and a surfactant. Herein, surfactants refer to molecules comprising a structural component of the LNPs. Additionally or alternatively, the surfactants described herein may not behave like detergents. For example, CTAB may behave like a detergent and not like a structural component of the LNP and may not be considered a surfactant in the context of the disclosure.

In some examples, the lipids may comprise an ionizable lipid. In further examples, the ionizable lipid may be a tertiary amine. As one example the lipid nanoparticles may be comprised of 6-((2-hexyldecanoyl)oxy)-N-(6-((2-hexyldecanoyl)oxy) hexyl)-N-(4-hydroxybutyl) hexan-1-aminium (ALC), 1,2-distearoyl-sn-glycero-3-phosphocholine (DSPC), cholesterol, and DMG-PEG2000 at molar ratios of 50:10:38:2 and may be herein referred to as ALC-LNPs. As a further example the lipid nanoparticles may be comprised of dimethyldioctadecylammonium (Bromide Salt) (DDAB), Soy phosphatidylcholine (SoyPC), cholesterol, and 1,2-dimyristoyl-rac-glycero-3-methoxypolyethylene glycol-2000 (DMG-PEG2000) in molar ratios of 50:10:38:2 and may be herein referred to as DDAB-LNPs. It is understood that ALC and DDAB are non-limiting examples of ionizable lipids and LNPs may include other ionizable lipids such as, but not limited to, 1,2-dioleyloxy-3-dimethylaminopropane (DODMA), N-(4-carboxybenzyl)-N,N-dimethyl-2,3-bix (oleoyloxy) proan1-aminium (DOBAQ), and dipalmitoyl-rac-glycero-3-carboetaine (DPCB).

The API may be a biologic having therapeutic properties. For example, the biologic may be an oligonucleotide, a fraction of RNA or DNA, or antibody. The RNA or DNA may each be single stranded or double stranded and may include at least 10 bases. Types of RNA may include but are not limited to messenger RNA (mRNA), transfer RNA (tRNA), small interfering RNA (siRNA), microRNA (miRNA), RNA aptamers, and antisense RNA (asRNA). Types of DNA may include but are not limited to plasmid DNA, DNA aptamers, DNA acidzymes, antisense oligonucleotides, and antigene oligonucleotides.

The lipid nanoparticles may be formed by homogenizing an organic stream including the lipids with an aqueous stream including the API. A v/v ratio of organic stream to aqueous stream may be in a range of 1:3 to 3:1. The homogenization process, may include, but is not limited to, multi-inlet vortex mixing, high pressure homogenization, impinging jet mixing, flow through laminar microfluidic devices, and ultrasonication. Preparing the LNP suspension may not include adding the barrier matrix stabilizer. The barrier matrix stabilizer may not be included in the liquid before homogenization to form the LNPs. In this way, the barrier matrix stabilizer may not be included in the LNP. The resulting suspension of LNPs may be further diluted with an aqueous bath after homogenization at a v/v ratio in a range of 1:5 up to 1:20 (LNPs suspension: aqueous bath). In some examples, the aqueous bath may include a buffer system.

At 103, method 100 optionally includes adjusting a buffer system of the LNP suspension. As described above with respect to step 102. The LNP suspension may include an aqueous phase which includes a buffer system. Adjusting the buffer system may include adding buffer system components to the LNP suspension to adjust a buffering strength or pH. In an alternate example, adjusting a buffer system may include performing a buffer exchange. For example, tangential flow filtration may be used to remove the buffer salts present after step 102 and different buffer salts may be added to the LNP suspension.

The buffer system may be selected to maintain a pH of the LNP spray drying matrix mixture. The pH range may be selected based on the stable pH range for the LNP and the API. As one example, the buffer system may be selected for a pH range between 5 and 10. In further examples, the buffer system may be selected for a pH range between 5 and 8. In some examples, the buffer system may be an acetate buffer system, including acetic acid and acetate salt (e.g., sodium acetate). In an alternate example, the buffers system may be a citrate buffer system, including citric acid and a citrate salt (e.g., trisodium citrate). Additionally, the buffer system may be selected to synergistically stabilize the LNPs in combination with the matrix stabilizer excipient.

At 104, method 100 includes adding excipients to the LNP suspension to form a LNP matrix mixture. The excipients may include a barrier matrix stabilizer. The barrier matrix stabilizer may be added to the LNP suspension after the LNP suspension is formed and not to either of the liquid phases while forming the LNP suspension. The LNP matrix mixture may be comprised of the LNPs suspended in the liquid that is a mixture of organic and aqueous liquids along with a barrier matrix stabilizer and a buffer dissolved in the liquid surrounding the LNPs. The barrier matrix stabilizer may be a high molecular weight molecule. In some examples, the barrier matrix stabilizer may additionally have a high glass transition temperature and a capacity for hydrogen bonding. For example, the barrier matrix stabilizer may have a molecular weight greater than or equal to 1000 g/mole. In some examples, a glass transition temperature for the dry barrier matrix stabilizer may be greater than or equal to 120° C. In alternate examples, a glass transition temperature for the dry barrier matrix stabilizer may be greater than or equal to 200° C. Additionally, the barrier matrix stabilizer may also be an FDA approved excipient for injectable drug formulations.

The barrier matrix stabilizer may be a cyclodextrin. For example, the barrier matrix stabilizer may be one or more of an alpha, beta, and gamma cyclodextrin. As a further example, the barrier matrix stabilizer may be a substituted cyclodextrin. The barrier matrix stabilizer may be substituted beta-cyclodextrin. For example, the beta cyclodextrin may include one or more of 2-hydroxypropyl-beta-cyclodextrin, 2-hydroxyethyl-beta-cyclodextrin, dimethyl-beta-cyclodextrin, trimethyl-beta-cyclodextrin, 3-hydroxypropyl-beta-cyclodextrin, and sulfobutyl ether-beta-cyclodextrin. In one example, the barrier matrix stabilizer may be one or more of oral grade, parenteral grade, and biopharma grade. Additionally or alternatively, a molar substitution of the substituted beta-cyclodextrin may be in a range of 0.4 to 1.5 substituent (e.g., hydroxypropyl) groups per anhydroglucose unit. In some examples, the LNP matrix solution may not include additional matrix stabilizers which are low molecular weight (e.g., <1000 g/mol) and do not have a high glass transition temperature (e.g., <200° C.). Herein, matrix stabilizers refers to molecular excipients added for structural stability of the LNPs and may not include buffer excipients added to maintain a pH of the LNP suspension. Low molecular weight excipients of the LNP matrix mixture may be limited to a buffer system. For example, the LNP matrix mixture may not include monosaccharides, disaccharides or small sugar alcohols such as mannitol. Without being bound by theory, including a high molecular weight, high glass transition temperature, strong hydrogen bonding matrix stabilizer such as substituted beta-cyclodextrin may help to minimize interactions between dried LNP particles which cause irreversible particle aggregation, observed as increases in average particle size and polydispersity index (PDI) when spray dried LNPs are redispersed.

The barrier matrix stabilizer may better stabilize the LNP particles than smaller molecules which also form strong hydrogen bonds, such as monosaccharides or disaccharides and sugar alcohols. A greater molecular weight molecule may occupy more space on a molecular basis and have a higher propensity for weakly associated cross-linking, both of which may help to prevent interactions between LNPs. Further, a higher molecular weight excipient may be included in a matrix formulation as a higher weight fraction on a per-molar basis when a final injectable formulation is limited by a maximum osmolarity. Additionally, during spray drying, a higher molecular weight excipient may have a slower rate of diffusion than a smaller molecule excipient. The higher molecular weight excipient may form a particle skin during spray drying to protect the LNP from severe temperature and shear stresses.

Further, the high glass transition temperature of the barrier matrix stabilizer along with the high molecular weight may decrease a mobility of the LNPs in the matrix, making it harder for LNPs to interact with each other during the drying process. The strong hydrogen bonding of the barrier matrix stabilizer may offer further stabilization to the LNPs. Without being bound by theory, the barrier matrix stabilizer may hydrogen bond polar groups of the lipid membrane and replace the water which is lost during drying. This replacement may prevent the formation of a gel state which may help prevent cargo (e.g., API) leakage from the LNPs.

The excipients added to the LNP suspension may be added in amounts determined based on target properties of an injectable formulation to be prepared with the spray dried LNP matrix particles. Turning briefly to FIG. 2A, a method 200 is shown for determining a solid weight percent composition of the LNP matrix particles. At 202, method 200 includes determining a target dosage, pH, maximum injection volume, and a maximum osmolarity of the injectable formulation. As one example, a maximum acceptable injection volume may be a volume which may be comfortably delivered in a single injection. The injection may be in the form of a vaccine administered by a technician in a pharmacy, for example and may not be administered by an auto-injector. The vaccine may be administered by the technician using a syringe and hypodermic needle. For example, the desired volume may be 1 mL.

As one example, a maximum osmolarity may be less than or equal to 300 mOsm/L. In a further example, the maximum osmolarity may be less than or equal to 600 mOsm/L. A maximum osmolarity may be above 300 mOsm/L and may be increased as high as 600 mOsm/L for formulations demanding a high dosage and low concentration of API in the LNPs. In some examples, the determined osmolarity may be greater than a minimum osmolarity. Injections below a minimum osmolarity may force water into cells and also cause patient discomfort. For example, a minimum osmolarity may be 100 mOsm/L. Increasing the osmolarity of an injection above the minimum osmolarity may include adding salts to the injectable solution. In some examples the maximum osmolarity may be in a range of 100 mOsm/L up to 300 mOsm/L. In further examples, the maximum osmolarity may be in a range of 100 mOsm/L up to 600 mOsm/L. Keeping the maximum osmolarity of an injectable formulation as low as possible without compromising dose or stability may help minimize discomfort caused by the injection.

A desired pH may be selected based on the delivery target of the injectable formulation. A pH of the injectable formulation for subcutaneous injection may be in a range of 4 to 9. A pH of the injectable formulation for intravenous or intramuscular injection may be in a range of 2-11. A desired pH may also be selected based on a chemical stability of the LNPs. For example, a desired pH range may be in a range of 4-10, in a range of 4-9, or in a range of 5-8.

At 204, method 200 includes setting a buffer system concentration to maintain the formulation at the desired pH. The buffer system concentration may be high enough to provide buffering capacity to prevent pH from being outside of the desired range over the course of multiple days. As one example the buffer system concentration may be in a range of 10 mM up to and including 50 mM.

At 206, method 200 includes setting an amount of LNPs demanded to minimize a buffer:LNP ratio while maintaining LNP stability and to reach the target dosage at or below the maximum injection volume. LNP stability may not be maintained if LNP concentration is high enough to cause LNPs to irreversibly agglomerate, thereby increasing an average LNP diameter above the threshold diameter. A minimum amount of LNPs may be based on both the target dosage, the injection volume, a loading of API in the LNPs and an encapsulation efficiency of the LNPs.

At 208, method 200 includes determining a solid weight percent of barrier matrix stabilizer to effectively stabilize the LNPs during spray drying and resuspension while remaining below the maximum osmolarity when the set amount of LNPs and determined amount of buffer system are in the injection volume. Method 200 ends. In this way, the LNP spray drying matrix mixture may include a maximum amount of LNPs to maintain LNP stability and a minimum amount of buffer system to stabilize the pH of the solution and a remainder solids content of the LNP spray drying matrix solution to reach the target osmolarity may be comprised of the barrier matrix stabilizer. In this way the weight percent of the matrix stabilizer in the LNP spray drying matrix solution and the resulting LNP spray dried matrix particles may be based on the target osmolarity of the injectable formulation.

The relationship between a solids weight percent of the barrier matrix stabilizer, LNPs, and buffer system is shown in graph 250 in FIG. 2B. Graph 250 shows solids weight percent of the LNP matrix mixture as function of target osmolarity for a given injection volume and target dosage and pH. A first plot 252 corresponds to the solids weight percent of the barrier matrix stabilizer. A second plot 254 corresponds to the solids weight percent of the buffer system, and a third plot 256 corresponds to the solids weight percent of the LNPs. As shown in graph 250, when a maximum osmolarity is high, the solids weight percent of the barrier matrix stabilizer is concomitantly increased.

Further, the relationship between components of the spray dried LNP matrix particles may be balanced between increasing a HP-b-CD fraction to improve LNP resuspension (e.g., maintaining particle size and distribution as well as encapsulation efficiency) within the constraint of a maximum osmotic strength. As shown in FIG. 2B, when increasing a maximum osmolarity, a fraction of HP-b-CD may be increased while fractions of buffer and LNP may be concomitantly reduced. Equation 1 below describes how components of the spray dried LNP matrix particle formulation contribute to osmolarity.

$${C}_{max} \leq \frac{D}{V {x}_{a}} ( \frac{1}{\overline{{MW}_{m}}} \frac{{w}_{m}}{{w}_{lnp}} + \frac{z}{M {W}_{b}} r ) \qquad ( 1 )$$

Cₘₐₓ is the maximum osmolarity, D is the target dosage, V is maximum injection volume, xₐ is the mass fraction of active ingredient (API) in the LNPs, wₘ is the mass fraction of matrix in the spray dried matrix particles, wₗₙₚ is the mass fraction of LNPs in the spray dried matrix particles, w_{b} is the mass fraction of buffer salts in the spray dried matrix particles, r is equal to a ratio of w_{b} to wₗₙₚ

$$( e . g . , \frac{{w}_{b}}{{w}_{lnp}} ) ,$$

M̅W̅ₘ̅ is the average molecular weight of the matrix excipient, M̅W̅_̅{̅b̅}̅ is the average molecular weight of the buffer, and Z is equal to a number of ions attributed to each buffer salt molecule. For example, if sodium acetate is fully dissociated Z is equal to 2 and if sodium acetate is not dissociated Z is equal to 1, partial dissociation results in a value of Z between 1 and 2.

Of the variables of equation 1, wₘ and wₗₙₚ are tunable and remaining variables are either fixed by the target product (e.g., V), part of the optimized manufacturing process

$$( e . g . , \frac{{w}_{b}}{{w}_{lnp}} ) ,$$

or are material attributes (e.g., M̅W̅_̅{̅b̅}̅). If Cₘₐₓ increases, then wₘ/wₗₙₚ may increase by increasing wₘ and decreasing wₗₙₚ as provided in the mass balance of equations 2 and 3 shown below.

$${w}_{lnp} + {w}_{m} + {w}_{b} = 1 \qquad ( 2 )$$

$${w}_{m} = 1 - {w}_{lnp} ( 1 + r ) \qquad ( 3 )$$

As shown by equations 2 and 3, for a fixed value of r, wₘ increases by decreasing wₗₙₚ.

Table 1 below shows ranges of solid weight composition of LNPs and excipients included in the dried LNP matrix particles. It is assumed that ratios of the masses added to the LNP spray drying matrix mixture are maintained throughout the spray drying process. In some examples, the LNP spray drying matrix mixture may be comprised only of the solid components listed in Table 1 in addition to solvent. The solid weight percent may be chosen to include a maximum amount of the effective HP-b-CD without surpassing the target osmolarity.

TABLE 1
Percent by weight of solid components
of dried LNP matrix particles.

| Component | Weight percentage |
|---|---|
| LNP | 0.3%-10% |
| HP-b-CD | 40%-99% |
| Buffer system | 0.5%-50% |

In some examples, the LNPs may be included in range of 1% up to 6% by mass. In some examples, HP-b-CD may be included in a range of 40% up 75% by mass. In alternate examples, HP-b-CD may be included in a range of 75% up to 98% by mass. In some examples, buffer system may be included in range of 20% up to 50% by mass. In further examples, buffer system may be included in amass of 1% up to 20% by mass. A mass percent of the buffer system may be set by the desired ratio of buffer system LNPs. For example, a mass ratio of LNP:non-volatile buffer component may be in a range of 1:2 up to 1:8. For example, a mass of citrate or acetate in addition to counterions to achieve the desired pH is included in the mass of non-volatile buffer component. As a further example, a mass ratio of LNP:non-volatile buffer component may be in a range of 1:1 up to 1:20. A ratio of LNPs to buffer system may be independent of a maximum osmolality of the injectable formulation.

Returning now to FIG. 1, method 100 continues to 106 and includes spray drying the LNP matrix mixture to obtain dried LNP matrix particles. The spray drying process removes solvent and the percent composition by weight of the dried LNP matrix particles is equivalent to weight percent of solid components in the LNP spray drying matrix mixture as described above. Spray drying may include spray drying at an inlet temperature and outlet temperature selected to reach a target dryness of the product while not subjecting the API to a temperature which may cause degradation and inactivation of the API. For example, spray drying may include spray drying at an outlet temperature in a range of 5° C. up to 45° C. In some examples, spray drying may include vacuum spray drying wherein the spray drying system is under reduced pressure (e.g., less than atmospheric pressure) during drying. For example, a drying chamber of the spray dryer may be at range of 0.05-1 bara. Vacuum spray drying may help to dry the LNP at lower temperatures to prevent degradation of the API.

When administration of the API is demanded, method 100 proceeds to 108 and includes mixing the dried LNP matrix particles in a vehicle to prepare the LNP injectable formulation. For example, the vehicle may be an aqueous vehicle. The LNPs of the LNP matrix particles may be suspended in the aqueous vehicle while the buffer system and barrier matrix stabilizer dissolve in the aqueous vehicle. For example, the LNP matrix particles may be mixed in pharmaceutical grade water. The dried LNP matrix particles including the barrier matrix stabilizer, such as HP-b-CD, may increase in average size, as evidenced by intensity weighted hydrodynamic diameter (Z-avg) and PDI less than dried LNP matrix particles that do not include the barrier matrix stabilizer. Further, the dried LNP matrix particles including the barrier matrix stabilizer may have a greater encapsulation efficiency upon resuspension than dried LNP matrix particles that do not include the barrier matrix stabilizer.

As described above, it is desired that the spray dried LNP matrix particles are below a threshold Z-avg (e.g., hydrodynamic diameter) and PDI when resuspended. Without being bound by theory, the LNPs may decrease in therapeutic effectiveness if the particle size and PDI are above the threshold amounts. As one example the threshold hydrodynamic diameter may be 200 nm and the threshold PDI may be 0.2.

Turning now to FIG. 3, a chart 300 is shown, comparing diameter and PDI of different LNP matrix particles before and after spray drying. A left axis 302 of chart 300 corresponds to diameter expressed as the intensity weighted mean hydrodynamic diameter (Z-avg) and a right axis 304 corresponds to PDI as determined from the Z-avg distribution. Unfilled bars show Z-avg of the LNPs in the LNP matrix solution before spray drying and filled circles show the PDI of the LNP matrix solution before spray drying. Filled bars show the Z-avg of the dried LNP matrix particles resuspended in an aqueous solvent (e.g., water) and open circles correspond to the PDI of the resuspended LNP matrix particles. Z-avg and PDI may be measured by dynamic light scattering (DLS) method accounting for viscosity of the liquid phase of the measured LNP suspension.

The portion of chart 300 within box 306 corresponds to LNP matrix particles including HP-b-CD as a barrier matrix stabilizer. The portion of chart 300 not within box 306 corresponds to LNP matrix particles including either a disaccharide (trehalose) or an amino acid (arginine) as the conventional matrix stabilizer. The chart includes data for each of the conventional matrix stabilizer and barrier matrix stabilizer including DDAB-LNPs from 0.3 wt. % up to 5.0 wt. %, matrix stabilizer from 25 wt. % up to 75 wt. % and the remainder of the weight made up by buffer systems of either citrate or acetate. As shown in chart 300, including the barrier matrix stabilizer more consistently results in smaller change in Z-average and PDI for resuspension after spray drying than either trehalose or arginine. Even though trehalose and arginine may exhibit hydrogen bonding, they are not as high in molecular weight or in glass transition temperature as HP-b-CD and do not stabilize the LNPs as well as HP-b-CD.

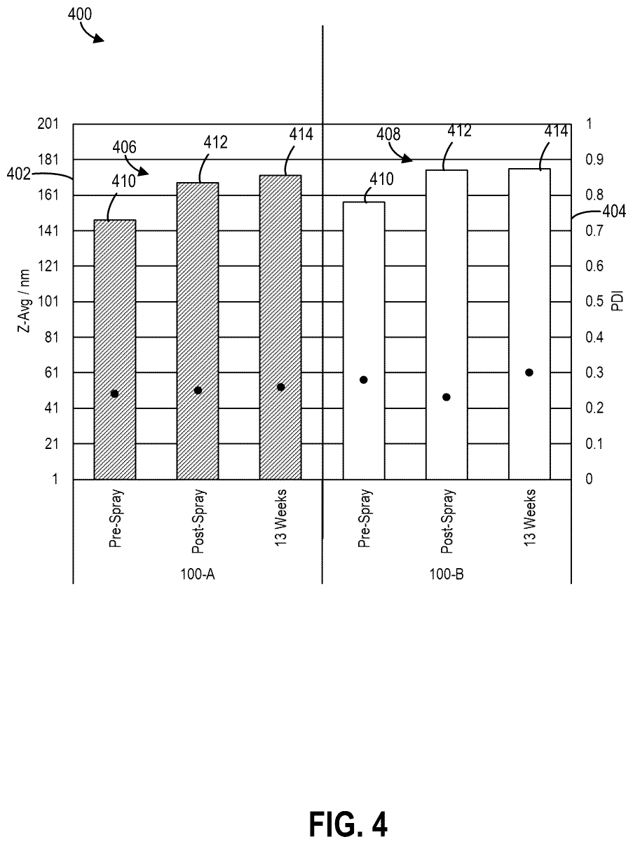

As discussed above, dried LNP matrix particles may be desired for enabling long term ambient temperature storage of LNPs. LNPs may be stored as a dry powder and reconstituted in an aqueous vehicle prior to injection. When reconstituted, the LNPs form a suspension and suspension of the LNPs as unagglomerated nanoparticles is desired. This may be different from protein therapeutics which are conventionally dissolved in the vehicle to form a solution. Turning now to FIG. 4, a chart 400 comparing the physical stability of dried ALC-LNP matrix particles stored for thirteen weeks under ambient conditions (e.g., ambient temperature, pressure, and humidity) is shown. A left axis 402 of chart 400 corresponds to Z-avg and a right axis 404 corresponds to PDI. Bars of chart 400 correspond to left axis 402 and circles correspond to right axis 404. Chart 400 shows a first data set 406 and a second data set 408. Both the first and second data sets correspond to LNP matrix particles including 3.5 wt. % LNPs, 75 wt. % HP-b-CD and 21.5 wt. % citrate buffer system. A first bar 410 of each set corresponds to Z-avg of the pre-spray dried LNP matrix solution and a circle positioned within the bar corresponds to the pre-spray dried PDI. A second bar 412 of each set corresponds to Z-avg of resuspended post-spray dried LNP matrix particles and a circle within the bar corresponds to a PDI of the post-spray dried LNP matrix particles. The post-spray dried LNP matrix particles are resuspended and measured within 24 hours of spray drying. A third bar 414 of each set corresponds to Z-avg of LNP matrix particles resuspended after 13 weeks of storage of the dried LNP matrix particles at ambient conditions, and an open circle positioned within the bar corresponds to a PDI of said resuspended LNP matrix particles.

As shown in FIG. 4, the effectiveness of HP-b-CD as a barrier matrix stabilizer extends to different types of LNPs besides the DDAB-LNPs. Additionally, the similarity of first data set 406 and second data set 408 shows that the effects of HP-b-CD as the barrier matrix stabilizer are reproducible. Further, FIG. 4 shows that there is only a small change in Z-avg and PDI after storage of the dried LNP matrix particles for thirteen weeks at ambient conditions. The ambient stability of the dried particles is desired for ease of storage and transportation of the LNP matrix particles.

In addition to physical stability, it is desired that the LNP matrix particles maintain an encapsulation efficiency of the API during spray drying and subsequent resuspension. Herein, encapsulation efficiency is a fraction of API encapsulated in the LNPs divided by the total API (e.g., RNA) present in the sample. Migration of API from inside the LNPs to outside the LNPs decreases the measured encapsulation efficiency.

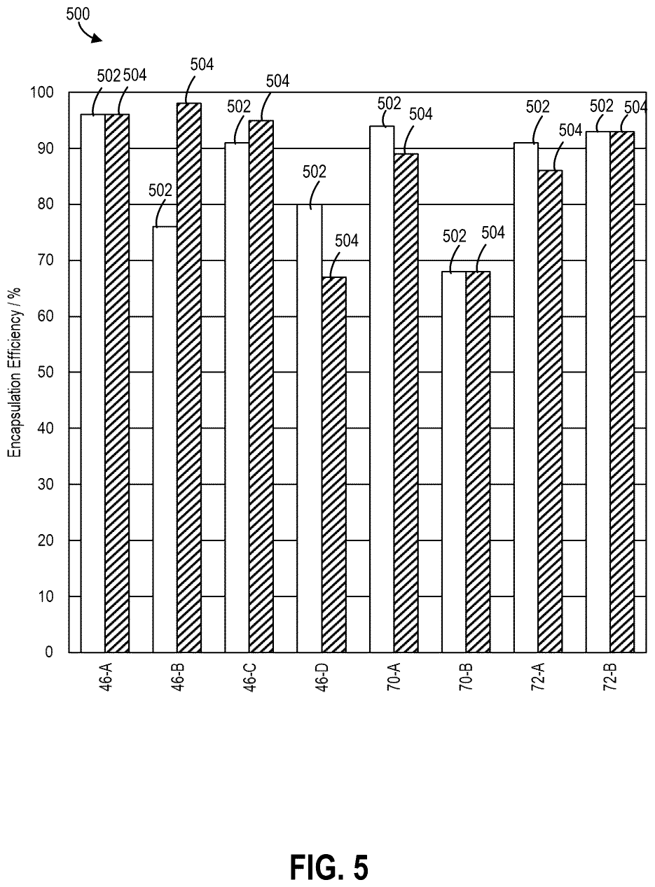

Turning now to FIG. 5, a chart 500 is shown comparing encapsulation efficiencies of pre-spray dried LNP matrix solution and post spray dried and resuspended dried LNP matrix particles. Left bars 502 correspond to pre-spray dried LNP matrix solution and right bars 504 correspond to resuspended post-spray dried LNP matrix particles. Each pair of bars corresponds to LNP matrix particles including 3.5 wt. % LNPs, 75 wt. % HP-b-CD and 21.5 wt. % citrate buffer system. The LNPs may be encapsulating an RNA API.

As shown in FIG. 5, on average, encapsulation efficiency in the LNP matrix particles including HP-b-CD as the barrier matrix stabilizer is maintained or not substantially (e.g., by less than 5%) decreased. Increases in encapsulation efficiency after spray drying shown in FIG. 5 may be artifacts of the measurement of encapsulation efficiency.

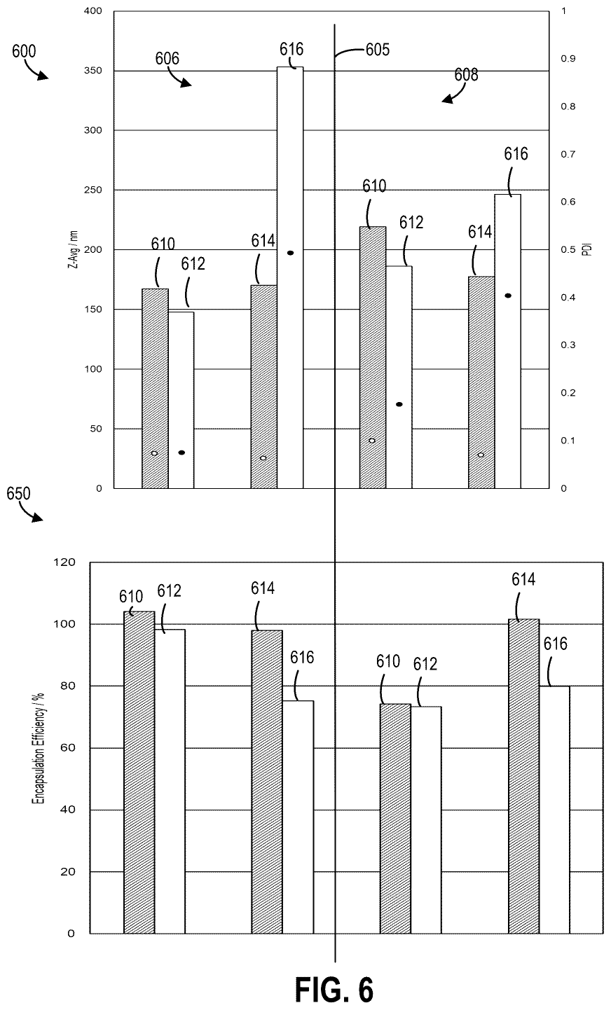

As discussed above, including a barrier matrix stabilizer, such as HP-b-CD in dried LNP matrix particles maintains a Z-avg, PDI, and encapsulation efficiency during spray drying, storage, and subsequent resuspension. The barrier matrix stabilizer may better maintain the desired physical properties of the LNPs than other, conventional matrix stabilizers, such as trehalose. Turning now to FIG. 6, a first chart 600 and second chart 650 comparing the dried LNP matrix particles including HP-b-CD and equivalent dried LNP matrix particles wherein the HP-b-CD is replaced with trehalose to the same target dose and osmolarity are shown. For a target dose of 100 μg/mL RNA and a 300 mOsm/L LNP matrix solution, the HP-b-CD LNP matrix particles include 1.2 wt. % LNPs, 95 wt. % HP-b-CD, and 3.8 wt. % citrate buffer system. Under the same target dose and osmolarity constraints, the trehalose LNP matrix particles include 4.3 wt. % LNPs, 0.81% trehalose, and 14.7 wt. % citrate buffer system. FIG. 6 also shows results of LNP matrix particles having a target dose of 100 μg/mL RNA and a 600 mOsm/L LNP matrix solution. When the target osmolarity is increased to 600 mOsm/L, the HP-b-CD matrix particles include 0.4 wt. % LNPs, 98 wt. % HP-b-CD, and 1.6 wt. % citrate buffer system and the trehalose LNP matrix particles include 1.6 wt. % LNPs, 93 wt. % trehalose, and 5.4 wt. % citrate buffer system. The above described compositions illustrate an advantage in the barrier matrix stabilizer. HP-b-CD may comprise a greater wt. % of the LNP matrix particles than trehalose under the osmolarity constraints because the molecular weight of HP-b-CD is much larger than that of trehalose. Having a greater wt. % of the barrier matrix stabilizer may be more effective at physically spacing LNPs out and decreasing the probability of particle-particle interactions which may lead to aggregation.

In both first chart 600 and second chart 650, a first data set 606 to the left of line 605 corresponds to the LNP matrix particles described above having a 300 mOsm/L target osmolarity. A second data set 608 to the right of line 605 corresponds to the LNP matrix particles described above having a 600 mOsm/L target osmolarity. First bars 610 and second bars 612 correspond to the HP-b-CD LNP matrix particles described above before spray drying and after spray drying, respectively. Circles positioned within the bars in first chart 600 correspond to the PDI. Third bars 614 and fourth bars 616 correspond to the trehalose LNP matrix particles described above before and after spray drying. Circles positioned within the bars in first chart 600 correspond to PDI.

As shown in first chart 600, before spray drying the LNP matrix particles including HP-b-CD are similar to the LNP matrix particles including trehalose in both Z-avg and PDI. However, during spray drying, the HP-b-CD is a better matrix stabilizer of the LNPs than trehalose. Even at the lowest target osmolarity, the LNP matrix particles including HP-b-CD exhibit no effective (e.g., >5%) increase in size or PDI when resuspended after spray drying. In comparison, when the osmolarity is limited to 300 mOsm/L, the trehalose LNP matrix particles nearly double in Z-avg and show a large increase in PDI. Trehalose performs slightly better in terms of maintaining physical properties when osmolarity is increased to 600 mOsm/L, but the increases in PDI and Z-avg after spray drying are still larger than the increases measured for the corresponding HP-b-CD LNP matrix particles.

As shown in second chart 650, the HP-b-CD LNPs exhibit little (<5%) to no decrease in encapsulation efficiency when the LNP matrix particles are prepared with 300 mOsm/L or 600 mOsm/L. The LNP matrix particles including trehalose decrease from close to 100% to close to 80% for both the 300 mOsm/l and the 600 mOsm/L formulations.

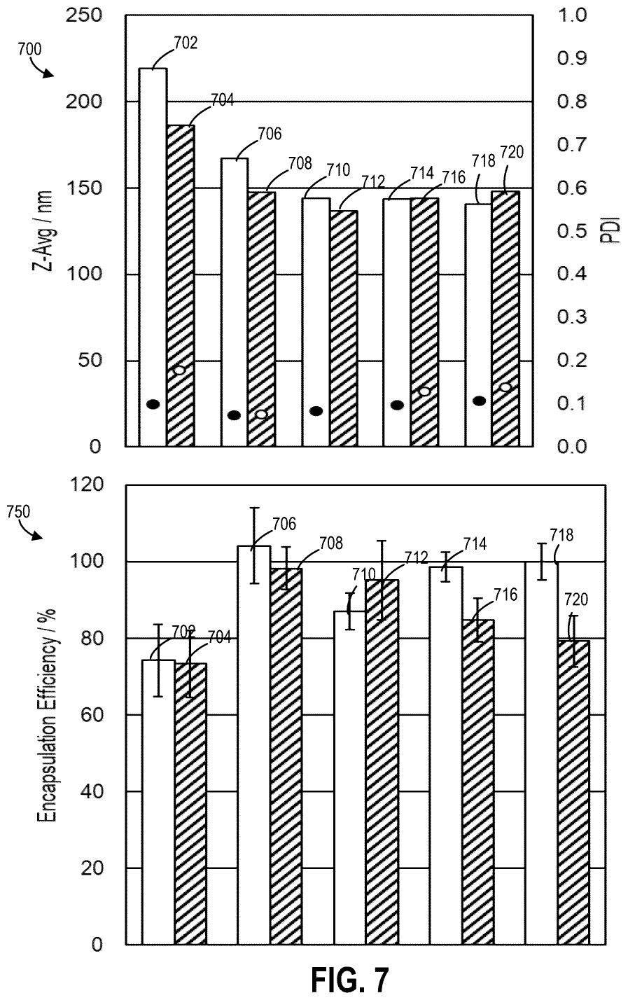

Conventionally, physical stability of spray dried LNP matrix particles may be increased by decreasing a weight percent of LNPs in the LNP matrix solution, thereby increasing a ratio of matrix stabilizer to LNPs. An unexpected benefit of including the barrier matrix stabilizer in the LNP matrix solution is that LNPs may be included at a higher weight percent. As shown below in FIG. 7, when including the barrier matrix stabilizer (e.g., HP-b-CD), the weight percent of LNPs in the LNP matrix particles may be increased up to at least 5.7% without negatively impacting physical stability or encapsulation efficiency. Increasing LNP wt. % in the LNP matrix particles may enable higher injection dosages at lower osmolarities.

FIG. 7 shows a first chart 700 comparing Z-avg (left axis) and PDI (right axis) for five different LNP matrix particles including HP-b-CD before and after spray drying. Bars of chart 700 correspond to the Z-avg and the circles positioned within the bar correspond to PDI of the same LNPs. A second chart 750 compares encapsulation efficiencies before and after spray drying for the same five LNP matrix particles including HP-b-CD. Each of the LNP matrix particles shown in FIG. 7 include ALC-LNPs and are prepared with target osmolarity of 600 mOsm/L. First bars 702 and second bars 704 correspond to LNP matrix particles including 0.4 wt. % LNPs, 98% HP-b-CD and 1.6 wt. % citrate buffer system, before and after spray drying respectively. Third bars 706 and fourth bars 708 correspond to LNP matrix particles including 1.2 wt. % LNPs, 95% HP-b-CD and 3.8 wt. %, before and after spray drying respectively. Fifth bars 710 and sixth bars 712 correspond to LNP matrix particles including 2.5 wt. % LNPs, 89 wt. % HP-b-CD and 8.5 wt. % citrate buffer system, before and after spray drying respectively. Seventh bars 714 and eighth bars 716 correspond to LNP matrix particles including 3.9 wt. % LNPs, 83 wt. % HP-b-CD and 13.1 wt. % citrate buffer system, before and after spray drying respectively. Ninth bars 718 and tenth bars 720 correspond to LNP matrix particles including 5.7 wt. % LNPs, 75 wt. % HP-b-CD and 19.3 wt. % citrate buffer system, before and after spray drying respectively.

As shown in first chart 700, even at the highest wt. % of LNPs the Z-avg and PDI are not significantly (by more than 5%) increased after spray drying and resuspension. The encapsulation efficiencies shown in second chart 750 are similarly relatively unchanged after spray drying for LNP matrix particles comprising up to 5.7 wt. % LNPs. In this way, HP-b-CD is an effective matrix stabilizer for producing spray dried LNP matrix particles that include a high percentage of LNPs and still maintain desired physical attributes when resuspended.

The technical effect of the method and formulations described herein is to stabilize LNPs during spray drying, such that physical attributes of the LNPs are maintained when resuspended. Recognizing the key attributes of a barrier matrix stabilizer include high molecular weight, high glass transition temperature, and ability to hydrogen bond. HP-b-CD is identified as an example barrier matrix stabilizer which meets the above characteristics and is approved for parenteral administration. Additionally, the formulations maintain a maximum amount of the barrier matrix stabilizer while recognizing that there is a maximum osmolarity for the prepared injections to maximize patient comfort.

The disclosure also provides support for a spray dried lipid nanoparticle matrix particles, comprising: lipid nanoparticles, a buffer system, and a barrier matrix stabilizer, wherein the barrier matrix stabilizer is included in a solid weight percent based on a maximum osmolarity and a maximum acceptable injectable volume of an injectable formulation comprising the spray dried lipid nanoparticle matrix particles. In a first example of the system, the barrier matrix stabilizer is 2-hydroxypropyl-beta-cyclodextrin (HP-b-CD). In a second example of the system, optionally including the first example, the HP-b-CD comprises between 40 wt. % and 99 wt. % of the spray dried lipid nanoparticle matrix particles. In a third example of the system, optionally including one or both of the first and second examples a ratio of lipid nanoparticles to buffer system is independent of the maximum osmolarity of the injectable formulation. In a fourth example of the system, optionally including one or more or each of the first through third examples, the buffer system is a citrate buffer system or an acetate buffer system. In a fifth example of the system, optionally including one or more or each of the first through fourth examples, the lipid nanoparticles encapsulate one or more of an antibody, oligonucleotide, DNA and RNA. In a sixth example of the system, optionally including one or more or each of the first through fifth examples, the lipid nanoparticles encapsulate one or more of mRNA, siRNA, miRNA, RNA aptamers, asRNA, and tRNA. In a seventh example of the system, optionally including one or more or each of the first through sixth examples, the lipid nanoparticles include tertiary amine ionizable lipids. In a eighth example of the system, optionally including one or more or each of the first through seventh examples, the spray dried lipid nanoparticle matrix particles are below a threshold hydrodynamic diameter and PDI when resuspended.

The disclosure also provides support for a method of spray drying lipid nanoparticles, comprising: preparing lipid nanoparticle suspension in a liquid, preparing a lipid nanoparticle matrix mixture by adding a barrier matrix stabilizer to the liquid, and spray drying the lipid nanoparticle matrix mixture to obtain spray dried lipid nanoparticle matrix particles, wherein a solid weight percent of the barrier matrix stabilizer is based on a maximum osmolarity of an injectable formulation comprising the spray dried lipid nanoparticle matrix particles. In a first example of the method, the barrier matrix stabilizer is 2-hydroxypropyl-beta-cyclodextrin. In a second example of the method, optionally including the first example, spray drying includes spray drying at an outlet temperature in a range of 5° C. up to 45° C. In a third example of the method, optionally including one or both of the first and second examples, the method further comprises adding the spray dried lipid nanoparticle matrix particles to a vehicle to prepare the injectable formulation at a target dose and the maximum osmolarity. In a fourth example of the method, optionally including one or more or each of the first through third examples, the maximum osmolarity is less than or equal to 600 mOsm/L. In a fifth example of the method, optionally including one or more or each of the first through fourth examples, the maximum osmolarity is less than or equal to 300 mOsm/L. In a sixth example of the method, optionally including one or more or each of the first through fifth examples, an encapsulation efficiency of the spray dried lipid nanoparticle matrix particles is not substantially decreased when the spray dried lipid nanoparticle matrix particles are resuspended. In a seventh example of the method, optionally including one or more or each of the first through sixth examples, preparing the lipid nanoparticle suspension in the liquid does not include adding barrier matrix stabilizer, and wherein the liquid includes water and an organic solvent. In a eighth example of the method, optionally including one or more or each of the first through seventh examples, the lipid nanoparticles include tertiary amine ionizable lipids.

The disclosure also provides support for a lipid nanoparticle matrix mixture for feeding into a spray dryer, comprising: lipid nanoparticles comprising 0.3%-10% of solids by weight, 2-hydroxypropyl-beta-cyclodextrin comprising 40%-99% of solids by weight, and a buffer system, comprising 0.5%-50% of solids by weight. In a first example of the system, spray dried lipid nanoparticle matrix particles formed from the lipid nanoparticle matrix mixture are included in an injectable formulation, and wherein a osmolarity of the injectable formulation is in a range of 300 mOsm/L up to 600 mOsm/L.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A spray dried lipid nanoparticle matrix particles, comprising:
lipid nanoparticles;
a buffer system; and
a barrier matrix stabilizer, wherein the barrier matrix stabilizer is included in a solid weight percent based on a maximum osmolarity and a maximum acceptable injectable volume of an injectable formulation comprising the spray dried lipid nanoparticle matrix particles.

2. The spray dried lipid nanoparticle matrix particles of claim 1, wherein the barrier matrix stabilizer is 2-hydroxypropyl-beta-cyclodextrin (HP-b-CD).

3. The spray dried lipid nanoparticle matrix particles of claim 2, wherein the HP-b-CD comprises between 40 wt. % and 99 wt. % of the spray dried lipid nanoparticle matrix particles.

4. The spray dried lipid nanoparticle matrix particles of claim 2, a ratio of lipid nanoparticles to buffer system is independent of the maximum osmolarity of the injectable formulation.

5. The spray dried lipid nanoparticle matrix particles of claim 1, wherein the buffer system is a citrate buffer system or an acetate buffer system.

6. The spray dried lipid nanoparticle matrix particles of claim 1, wherein the lipid nanoparticles encapsulate one or more of an antibody, oligonucleotide, DNA and RNA.

7. The spray dried lipid nanoparticle matrix particles of claim 1, wherein the lipid nanoparticles encapsulate one or more of mRNA, siRNA, miRNA, RNA aptamers, asRNA, and tRNA.

8. The spray dried lipid nanoparticle matrix particles of claim 1, wherein the lipid nanoparticles include tertiary amine ionizable lipids.

9. The spray dried lipid nanoparticle matrix particles of claim 1, wherein the spray dried lipid nanoparticle matrix particles are below a threshold hydrodynamic diameter and PDI when resuspended.

10. A method of spray drying lipid nanoparticles, comprising:
preparing lipid nanoparticle suspension in a liquid;
preparing a lipid nanoparticle matrix mixture by adding a barrier matrix stabilizer to the liquid; and
spray drying the lipid nanoparticle matrix mixture to obtain spray dried lipid nanoparticle matrix particles, wherein a solid weight percent of the barrier matrix stabilizer is based on a maximum osmolarity of an injectable formulation comprising the spray dried lipid nanoparticle matrix particles.

11. The method of claim 10, wherein the barrier matrix stabilizer is 2-hydroxypropyl-beta-cyclodextrin.

12. The method of claim 10, wherein spray drying includes spray drying at an outlet temperature in a range of 5° C. up to 45° C.

13. The method of claim 10, wherein the method further comprises adding the spray dried lipid nanoparticle matrix particles to a vehicle to prepare the injectable formulation at a target dose and the maximum osmolarity.

14. The method of claim 10, wherein the maximum osmolarity is less than or equal to 600 mOsm/L.

15. The method of claim 10, wherein the maximum osmolarity is less than or equal to 300 mOsm/L.

16. The method of claim 10, wherein an encapsulation efficiency of the spray dried lipid nanoparticle matrix particles is not substantially decreased when the spray dried lipid nanoparticle matrix particles are resuspended.

17. The method of claim 10, wherein preparing the lipid nanoparticle suspension in the liquid does not include adding barrier matrix stabilizer, and wherein the liquid includes water and an organic solvent.

18. The method of claim 10, wherein the lipid nanoparticles include tertiary amine ionizable lipids.

19. A lipid nanoparticle matrix mixture for feeding into a spray dryer, comprising:
lipid nanoparticles comprising 0.3%-10% of solids by weight;
2-hydroxypropyl-beta-cyclodextrin comprising 40%-99% of solids by weight; and
a buffer system, comprising 0.5%-50% of solids by weight.

20. The lipid nanoparticle matrix mixture of claim 19, wherein spray dried lipid nanoparticle matrix particles formed from the lipid nanoparticle matrix mixture are included in an injectable formulation, and wherein a osmolarity of the injectable formulation is in a range of 300 mOsm/L up to 600 mOsm/L.
